(19) United States
(12) Patent Application Publication
(10) Pub. No.: US 2003/0032676 A1
(43) Pub. Date: Feb. 13, 2003

(54) Drugs for treatment of cerebral injury and methods of use thereof

(76) Inventors: Harold K. Kimelberg, Albany, NY (US); Paul J. Feustel, Delmar, NY (US)

Correspondence Address: MINTZ, LEVIN, COHN, FERRIS,, GLOVSKY and POPEO, P.C., One Financial Center, Boston, MA, 02111, US

(21) Appl. No.: 10/133,618

(22) Filed: Apr. 26, 2002

Related U.S. Application Data
(60) Provisional application No. 60/311,270, filed on Aug. 9, 2001.
(60) Provisional application No. 60/332,128, filed on Nov. 21, 2001.
(60) Provisional application No. 60/362,287, filed on Mar. 7, 2002.

(51) Int. Cl. A61K 31/137

(52) U.S. Cl. 514/651

(57) ABSTRACT

Methods of treating stroke and conferring protection against cerebral injury in a subject following an ischemic event, wherein a tamoxifen compound is administered in an effective amount so as to confer protection on the population of cells. Treatable ischemic events include cerebrovascular disease or stroke, subarachnoid subhemorrhage, myocardial infarct, surgery and trauma.

### RELATED APPLICATIONS

This application claims the benefit of priority under 35 U.S.C. 119(e) to copending U.S. Provisional Application No. 60/311,270, filed on Aug. 9, 2001; 60/332,128, filed on Nov. 21, 2001; and 60/362,287, filed on Mar. 7, 2002; the entire contents of which are incorporated herein by reference.

### STATEMENT AS TO FEDERALLY SPONSORED RESEARCH

This invention was made with U.S. Government support under NS 35205. The government has certain rights in the invention.

### BACKGROUND OF THE INVENTION

Ischemia is an acute condition associated with an inadequate flow of oxygenated blood to a part of the body, caused by the constriction or blockage of the blood vessels supplying it. Ischemia occurs any time that blood flow to a tissue is reduced below a critical level. This reduction in blood flow can result from: (i) the blockage of a vessel by an embolus (blood clot); (ii) the blockage of a vessel due to atherosclerosis; (iii) the breakage of a blood vessel (a bleeding stroke); and (iv) the blockage of a blood vessel due to vasoconstriction such as occurs during vasospasms and possibly, during transient ischemic attacks (TIA) and following subarachnoid hemorrhage. Conditions in which ischemia occurs also include (i) myocardial infarction; (ii) trauma; and (iii) during cardiac and thoracic surgery and neurosurgery (blood flow needs to be reduced or stopped to achieve the aims of surgery). During myocardial infarct, heart stoppage or damage occurs which reduces blood flow to organs, and ischemia results. During various surgeries, blood flow reduction; clots; or air bubbles can lead to significant ischemic damage.

When an ischemic event occurs, there is a gradation of injury that arises from the ischemic site. The cells at the site of blood flow restriction, undergo necrosis and form the core of a lesion. A penumbra is formed around the core where the injury is not immediately fatal but progresses slowly toward cell death. This progression to cell death may be reversed upon reestablishing blood flow (reperfusion) within a short time of the ischemic event.

Focal ischemia encompasses cerebrovascular disease (stroke), subarachnoid hemorrhage (SAH) and trauma. Stroke is the third leading cause of morbidity in the United States, with over 500,000 cases per year, including 150,000 deaths annually. Post-stroke sequelae are mortality and debilitating chronic neurological complications which result from neuronal damage for which prevention or treatment are not currently available.

Following a stroke, the core area shows signs of cell death, but cells in the penumbra remain alive for a time, although malfunctioning, and will in several days resemble the necrotic core. The neurons in the penumbra seem to malfunction in a graded manner with respect to regional blood flow. As the blood flow is depleted, neurons fall electrically silent, their ionic gradients decay, the cells depolarize and then they die. Endothelial cells and astrocytes of the brain capillaries undergo swelling and the luminal diameter of the capillaries may decrease. Associated with these events, the blood brain barrier appears to be disrupted, and an inflammatory response follows which further interrupts blood flow and the access of cells to oxygen.

Despite the frequency of occurrence of ischemia (including stroke) and despite the serious nature of the outcome for the patient, treatments for these conditions have proven to be elusive. There are two basic approaches that have been undertaken to rescue degenerating cells in the penumbra. The first and most effective approach to date has been the identification of blood clot dissolvers that bring about rapid removal of the vascular blockage that restricts blood flow to the cells. Recombinant tissue plasminogen activator (TPA) has been approved by the Federal Drug Administration for use in dissolving clots that cause ischemia in thrombotic stroke. Nevertheless, adverse side effects are associated with the use of TPA. For example, a consequence of the breakdown of blood clots by TPA treatment is cerebral hemorrhaging that results from blood vessel damage caused by the ischemia. A second approach to treating degenerating cells deprived of oxygen is to protect the cells from damage that accumulates from the associated energy deficit. To this end, glutamate antagonists and calcium channel antagonists have been most thoroughly investigated. None of these have proven to be substantially efficacious but they are still in early clinical development.

### SUMMARY OF THE INVENTION

The present invention relates to the discovery that cerebral injury can be prevented or minimized by administration of certain neuroprotective compounds, and thus are beneficial in treating ischemic/hypoxic related conditions, e.g., stroke or heart attack in humans. In particular, it has been found that effective dosages of tamoxifen are useful as a neuroprotective agent, and is therefore valuable in the treatment of a variety of various ischemic/hypoxic related conditions such as ischemia-reperfusion injury, congestive heart failure, cardiac arrest and myocardial infarction such as due to coronary artery blockage. Tamoxifen has been found to protect against ischemic/hypoxic related cerebral injury.

The invention further includes neuroprotective agents containing tamoxifen, e.g., in an amount effective for neuroprotection, and a pharmaceutically acceptable carrier. Also included are kits for treating patients at risk of cerebral injury, e.g., from stroke, containing in one or more containers, an effective amount of tamoxifen, a pharmaceutically acceptable carrier, and instructions for use.

Another embodiment of the invention relates to method of treating or preventing cerebral hypoxic or ischemic damage in a subject, comprising administering to a subject in need thereof an effective amount of an agent which modulates cerebral hypoxic or ischemic damage such that cerebral cells are protected from cell death.

The invention addresses the need for effective treatments for stroke and other forms of ischemia that are safe, and may be administered preventatively to men and women who are susceptible to such conditions, and may further be used after the ischemia has occurred so as to protect cells from progressive degeneration that is initiated by the ischemic event.

These and other objects of the present invention will be apparent from the detailed description of the invention provided below.

### BRIEF DESCRIPTION OF THE DRAWING

FIG. 1 shows tamoxifen effectiveness in reversible MCAo, further detail of which is set forth in the Examples.

FIG. 2 shows that tamoxifen does not affect cerebral blood flow in the ischemic core, further detail of which is set forth in the Examples.

FIG. 3 shows the effectiveness of the invention in reducing infarct volume after permanent MCAo, further detail of which is set forth in the Examples.

FIG. 4 shows the tamoxifen dose response when given three hours after permanent MCAo, further detail of which is set forth in the Examples.

FIG. 5 shows how tamoxifen reduces EAA release during ischemia, further detail of which is set forth in the Examples.

FIG. 6 shows how tamoxifen inhibits nNOS during ischemia, further detail of which is set forth in the Examples.

FIG. 7 shows how tamoxifen reduces nitrotyrosine levels after rMCAo, further detail of which is set forth in the Examples.

FIG. 8 shows how tamoxifen inhibits lipid peroxidation during ischemia, further detail of which is set forth in the Examples.

### DETAILED DESCRIPTION OF THE INVENTION

The features and other details of the invention will now be more particularly described with reference to the accompanying drawings and pointed out in the claims. Particular embodiments described herein are shown by way of illustration and not as limitations of the invention. The principal features of this invention can be employed in various embodiments without departing from the scope of the invention. All parts and percentages are by weight unless otherwise specified.

Definitions

For convenience, certain terms used in the specification, examples, and appended claims are collected here. Unless otherwise defined, all technical and scientific terms used have the same meaning as commonly understood by one of ordinary skill in the art to which this invention pertains. However, to the extent that these definitions vary from meanings circulating within the art, the definitions below control.

“Ischemia” or “ischemic insult” includes the decrease or cessation of blood flow, e.g., in the brain.

“Hypoxia” includes the deficiency in the amount of oxygen reaching body tissues, e.g., the brain.

“Hypoxia or ischemic-related injury” includes cerebral injury.

“Reperfusion” includes the restoration of blood flow to an organ or tissue that has had its blood supply cut off, as after a heart attack or stroke.

“Oxidative stress” includes low O₂ conditions, e.g., that occur when there is an excess of free radicals, a decrease in antioxidant levels, or both; such as under ischemic or hypoxic conditions, and with reperfusion.

“Necrosis” includes cell or tissue death through injury or disease, particularly in a localized area of the body such as the myocardium.

“Apoptosis” refers to programmed cell death.

“Beta blockers” include agents such as atenolol, metoprolol, and propranolol, which act as competitive antagonists at the adrenergic beta receptors. Such agents also include those more selective for the cardiac (beta-1) receptors which allows for decreased systemic side effects. Beta blockers reduce the symptoms connected with hypertension, cardiac arrhythmias, migraine headaches, and other disorders related to the sympathetic nervous system. Beta blockers also are sometimes given after heart attacks to stabilize the heartbeat. Within the sympathetic nervous system, beta-adrenergic receptors are located mainly in the heart, lungs, kidneys, and blood vessels. Beta blockers compete with the nerve-stimulating hormone epinephrine for these receptor sites and thus interfere with the action of epinephrine, lowering blood pressure and heart rate, stopping arrhythmias, and preventing migraine headaches.

“Cerebral injury” includes any chronic or acute pathological event involving the brain and/or associated tissue, including ischemic insult, ischemia-reperfusion injury; cerebrovascular accident (stroke); congestive heart failure; cardiac arrest; myocardial infarction; cardiotoxicity caused by compounds such as drugs (e.g., doxorubicin, herceptin, thioridazine and cisapride); cardiac damage due to parasitic infection (bacteria, fungi, rickettsiae, and viruses, e.g. syphilis, chronic Trypanosoma cruzi infection); fulminant cardiac amyloidosis; heart surgery; heart transplantation; and traumatic cardiac injury (e.g., penetrating or blunt cardiac injury, aortic valve rupture).

“Subject” includes living organisms such as humans, monkeys, cows, sheep, horses, pigs, cattle, goats, dogs, cats, mice, rats, cultured cells therefrom, and transgenic species thereof. Preferably, the subject is a human. Administration of the compositions of the invention to a subject to be treated can be carried out using known procedures, at dosages and for periods of time effective to treat the condition in the subject. An effective amount of the therapeutic compound necessary to achieve a therapeutic effect may vary according to factors such as the age, sex, and weight of the subject, and the ability of the therapeutic compound to treat the foreign agents in the subject. Dosage regimens can be adjusted to provide the optimum therapeutic response. For example, several divided doses may be administered daily or the dose may be proportionally reduced as indicated by the exigencies of the therapeutic situation.

“Substantially pure” includes compounds, e.g., drugs, proteins or polypeptides that have been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis or HPLC analysis. A compound, e.g., a protein, is also substantially purified when it is essentially free of naturally associated components or when it is separated from the native contaminants which accompany it in its natural state. Included within the meaning of the term “substantially pure” are compounds, such as proteins or polypeptides, which are homogeneously pure, for example, where at least 95% of the total protein (by volume, by wet or dry weight, or by mole percent or mole fraction) in a sample is the protein or polypeptide of interest.

“Administering” includes routes of administration which allow the compositions of the invention to perform their intended function, e.g., treating or preventing cerebral injury caused by hypoxia or ischemia. A variety of routes of administration are possible including, but not necessarily limited to parenteral (e.g., intravenous, intraarterial, intramuscular, subcutaneous injection), oral (e.g., dietary), topical, nasal, rectal, or via slow releasing microcarriers depending on the disease or condition to be treated. Oral, parenteral and intravenous administration are preferred modes of administration. Formulation of the compound to be administered will vary according to the route of administration selected (e.g., solution, emulsion, gels, aerosols, capsule). An appropriate composition comprising the compound to be administered can be prepared in a physiologically acceptable vehicle or carrier and optional adjuvants and preservatives. For solutions or emulsions, suitable carriers include, for example, aqueous or alcoholic/aqueous solutions, emulsions or suspensions, including saline and buffered media, sterile water, creams, ointments, lotions, oils, pastes and solid carriers. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers (See generally, Remington's Pharmaceutical Science, 16th Edition, Mack, Ed. (1980)).

“Effective amount” includes those amounts of tamoxifen which allow it to perform its intended function, e.g., treating or preventing, partially or totally, cerebral injury caused by hypoxia or ischemia as described herein. The effective amount will depend upon a number of factors, including biological activity, age, body weight, sex, general health, severity of the condition to be treated, as well as appropriate pharmacokinetic properties. For example, dosages of the active substance may be from about 0.01 mg/kg to about 500 mg/kg, advantageously from about 0.1 mg/kg to about 100 mg/kg, more advantageously from about 10 to 30 mg/kg, and even more advantageously at about 10 to 20 mg/kg. Alternately the tamoxifen compound is administered at a dosage to provide a tamoxifen concentration in blood serum of from about 5 to 100 μM, about 5 to 20 μM, about 10 to 20 μM, or about 5 to 10 μM. A therapeutically effective amount of the active substance can be administered by an appropriate route in a single dose or multiple doses. Further, the dosages of the active substance can be proportionally increased or decreased as indicated by the exigencies of the therapeutic or prophylactic situation.

“Parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration includes, but is not limited to, subcutaneous, intraperitoneal, intramuscular, intrasternal injection, and kidney dialytic infusion techniques.

“Pharmaceutically acceptable carrier” includes any and all solvents, dispersion media, coatings, antibacterial and antifungal agents, isotonic and absorption delaying agents, and the like which are compatible with the activity of the compound and are physiologically acceptable to the subject. An example of a pharmaceutically acceptable carrier is buffered normal saline (0.15M NaCl). The use of such media and agents for pharmaceutically active substances is well known in the art. Except insofar as any conventional media or agent is incompatible with the therapeutic compound, use thereof in the compositions suitable for pharmaceutical administration is contemplated. Supplementary active compounds can also be incorporated into the compositions.

“Pharmaceutically acceptable esters” includes relatively non-toxic, esterified products of therapeutic compounds of the invention. These esters can be prepared in situ during the final isolation and purification of the therapeutic compounds or by separately reacting the purified therapeutic compound in its free acid form or hydroxyl with a suitable esterifying agent; either of which are methods known to those skilled in the art. Acids can be converted into esters according to methods well known to one of ordinary skill in the art, e.g., via treatment with an alcohol in the presence of a catalyst.

“Additional ingredients” include: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, e.g., in Remington's Pharmaceutical Sciences.

“Unit dose” includes a discrete amount of tamoxifen comprising a predetermined amount of the active ingredient.

Tamoxifen is shown effective as a human neuroprotectant against ischemic/hypoxic related conditions in the examples below, using rat models of permanent middle cerebral artery occlusion (MCAo). This model is well established as best resembling a human in vivo ischemic event, i.e., stroke. The experimental occlusion of the middle cerebral artery, as detailed below, causes a large unilateral ischemic area that typically involves the basal ganglion and frontal, parietal, and temporal cortical areas.

Tamoxifen, or (Z)-2-[4-(1,2-diphenyl-1-butenyl)phenoxy]-N,N-dimethylethanamine, is a polycyclic nonphenolic compound having the structure:

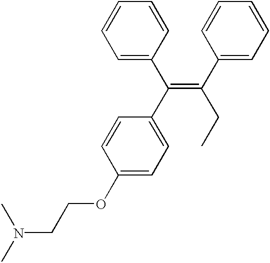

“Tamoxifen” includes derivatives of tamoxifen including side chain primary alcohol derivatives thereof; tamoxifen metabolites, e.g., human in vivo metabolites including and N-desmethyl tamoxifen and 4-hydroxytamoxifen; and salts thereof, e.g., tamoxifen citrate. It is not intended to include derivatives comprising a terminal phenolic ring substituent. Others have taught polycyclic phenolic compounds as neuroprotective, e.g., as disclosed in U.S. Pat. No. 6,319,914, but those teachings explicitly state compounds not having a phenolic structure on a terminal ring are not cytoprotective. It also includes structurally related compounds such as toremifine and idoxifene (see, e.g., MacGregor, J. I and Jordan, V. C Basic Guide to the Mechanisms of Antiestrogen Action. Pharmacological Reviews 50:151-196, 1998).

According to the invention, tamoxifen is effective in reducing the adverse effects of an ischemic event such as cerebrovascular disease, subarachnoid hemorrhage, or trauma. Accordingly, the compound is administered as soon as possible after initiation of the ischemic insult or event and preferably within 12 hours, more particularly, within 11 hours, more particularly, 8 hours, more particularly, 6 hours, more particularly, 4 hours, more particularly, within 3 hours following the event or the onset of reperfusion. Most preferably, for maximal effect, the compound is administered within one hour, or between one and three hours after, or between one and two hours after, the ischemic insult or the onset of reperfusion.

The invention also encompasses methods of treating or preventing cerebral injury caused by hypoxia or ischemia in a subject, wherein tamoxifen is administered such that hypoxia or ischemic-related cerebral injury is prevented or decreased, e.g., within one hour, or between one and three hours after, the ischemic insult or the onset of reperfusion. In certain embodiments, the tamoxifen is administered at a concentration of less than about 10 μM. In other embodiments, the tamoxifen is administered at a concentration in the range of about 10 nM to about 100 μM, about 10 nM to about 1 μM, 100 nM to about 10 μM, and 100 nM to about 500 nM. In still other embodiments of the invention, the tamoxifen is administered such that the concentration of tamoxifen is in the range of 5 μM to about 100 μM. Most desirably, the tamoxifen compound is administered at a dosage to provide a tamoxifen concentration in blood serum of from about 5 to 100 μM, about 5 to 20 μM, about 10 to 20 μM, or about 5 to 10 μM.

Tamoxifen may also be administered as a function of the subject's body weight. In some embodiments of the invention, tamoxifen is administered at a concentration of between about 1 g/kg to 1 g/kg of a subject's body weight, including less than 500 mg/kg, 250 mg/kg, 100 mg/kg, 20 mg/kg, 10 mg/kg, 5 mg/kg, 2 mg/kg, 1 mg/kg, 500 kg/kg, 250 μg/kg, 100 μg/kg, 10 μg/kg, 5 μg/kg, 2 μg/kg or 1 82 g/kg. More preferably the dosage range is about 1 mg/kg to 20 mg/kg, more preferably 5 mg/kg to 20 mg/kg. In some embodiments, tamoxifen is administered in combination with other compounds, such as anti-platelet drugs, anti-coagulant drugs, beta blockers, and anti-thrombotic drugs.

The invention still further encompasses methods of preventing stroke in a subject (e.g., a human) suffering from heart failure, by treating a subject with tamoxifen in a pharmaceutically acceptable carrier. In some embodiments, the tamoxifen is administered to the subject at a concentration of less than about 10 μM. The tamoxifen may be administered prior to, or concomitant with, a surgical procedure that may increase the likelihood of a stroke in the patient. In one embodiment, the procedure is balloon angioplasty. Other procedures include coronary artery bypass surgery and valve replacement surgery. The tamoxifen may be administered prior to, concomitant with, or after anti-thrombogenic agents (e.g., coumadin).

Excitatory amino acid (EAA) release that starts coincidentally with the onset of brain ischemia is widely believed to be a factor in stroke-induced damage. We have found that blockade of volume-regulated anion channels (VRACs) causes a reduction in ischemia-induced EAA release. Most VRAC inhibitors do not cross the blood-brain-barrier (BBB) and can only be examined with local application into brain tissue. In contrast, tamoxifen both potently blocks VRACs in vitro and readily permeates the BBB. We have also found tamoxifen to be a potent inhibitor of neuronal nitric oxide synthase, a potential source of damaging peroxynitrite. Other evidence indicates tamoxifen is also a potent oxygen radical scavenger in brain tissue. Thus, tamoxifen has multiple mechanisms of action, all of which could confer neuroprotection both during and after ischemia.

In certain embodiments, tamoxifen may be administered prior to a predicted ischemic event. This may happen e.g., when a subject has already had a stroke. In this case, the subject will have an increased probability of experiencing a second stroke. Subjects who are susceptible to transient ischemic attacks also have an increased risk of a stroke. Subjects who suffer a subarachnoid hemorrhage may experience further ischemic events induced by vasospasms that constrict the blood vessels. Subjects who experience trauma to organs such as the brain are also susceptible to an ischemic event. These situations exemplify circumstances when a subject would benefit from pretreatment with tamoxifen. Such pretreatment may be beneficial in reducing the adverse effects of a future ischemic event when administered in the short term, such as within 24 hours before the event or in the long term, where administration begins immediately after an event such as a stroke and continues prophylactically for an extended period of time. An example of time of administration for prophylactic use may extend from days to months depending of the particular susceptibility profile of the individual. In these circumstances, a course of at least one dose of tamoxifen may be administered over time so that an effective dose is maintained in the subject. For short term treatments, parenteral administration may be used as an alternative to the delivery of a dose by any of the routes specified below.

The tamoxifen of the invention may be a component of a pharmaceutical composition, which may also comprise buffers, salts, other proteins, and other ingredients acceptable as a pharmaceutical composition. The invention also includes a modified form of tamoxifen, which is capable of preventing or reducing hypoxic/ischemic cerebral injury as described herein.

Free radicals generated by ischemic or hypoxic conditions is believed to be a significant cause of cerebral damage leading to cell death. As such, administration of tamoxifen, administered in vivo, e.g., in non-toxic dosages, is an effective treatment for inhibiting or preventing oxidative stress free radical damage, either by tamoxifen-mediated free radical scavenging, or by inhibition of free radical generation.

In another embodiment, the invention relates to methods of treating or preventing cerebral oxidative stress in a subject, e.g., a human subject, wherein an effective amount of an agent which modulates cerebral oxidative stress, e.g., tamoxifen, is administered to a subject in need thereof such that the cells which are subject to oxidative stress are protected from cell death. In another aspect of the invention, tamoxifen has been found to modulate free radical damage caused by oxidative stress. As such, administration of tamoxifen, administered in vivo, is an effective treatment for inhibiting or preventing oxidative stress free radical damage.

The structure of the therapeutic compounds of this invention may include asymmetric carbon atoms. It is to be understood accordingly that the isomers (e.g., enantiomers and diastereomers) arising from such asymmetry are included within the scope of this invention. Such isomers can be obtained in substantially pure form by classical separation techniques and by sterically controlled synthesis. For the purposes of this application, unless expressly noted to the contrary, a therapeutic compound shall be construed to include both the R or S stereoisomers at each chiral center. In certain embodiments, an therapeutic compound of the invention comprises a cation. If the cationic group is hydrogen, H⁺, then the therapeutic compound is considered an acid. If hydrogen is replaced by a metal ion or its equivalent, the therapeutic compound is a salt of the acid. Pharmaceutically acceptable salts of the therapeutic compound are within the scope of the invention, e.g., pharmaceutically acceptable alkali metal (e.g., Li⁺, Na⁺, or K⁺) salts, ammonium cation salts, alkaline earth cation salts (e.g., Ca²⁺, Ba²⁺, Mg²⁺), higher valency cation salts, or polycationic counter ion salts (e.g., a polyammonium cation). (See, e.g., Berge et al. (1977) “Pharmaceutical Salts”, J Pharm. Sci. 66:1-19). It will be appreciated that the stoichiometry of an anionic compound to a salt-forming counter ion (if any) will vary depending on the charge of the anionic portion of the compound (if any) and the charge of the counter ion. Preferred pharmaceutically acceptable salts include a sodium, potassium or calcium salt, but other salts are also contemplated within their pharmaceutically acceptable range.

The invention encompasses the preparation and use of pharmaceutical compositions comprising a compound useful for the prevention or reduction of hypoxic/ischemic cerebral injury as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a pharmaceutically acceptable ester or salt, such as in combination with a physiologically-acceptable cation or anion, as is well known in the art. Further, the tamoxifen may contain pharmacologically acceptable additives (e.g., carrier, excipient and diluent), stabilizers or components necessary for formulating preparations, which are generally used for pharmaceutical products, as long as it does not adversely affect the efficacy of the preparation, e.g., in decreasing or inhibiting ischemia or reperfusion injury.

Examples of additives and stabilizers include saccharides such as monosaccharides (e.g., glucose and fructose), disaccharides (e.g., sucrose, lactose and maltose) and sugar alcohols (e.g., mannitol and sorbitol); organic acids such as citric acid, maleic acid and tartaric acid and salts thereof (e.g., sodium salt, potassium salt and calcium salt); amino acids such as glycine, aspartic acid and glutamic acid and salts thereof (e.g., sodium, calcium or potassium salt); surfactants such as polyethylene glycol, polyoxyethylene-polyoxypropylene copolymer and polyoxyethylenesorbitan fatty acid ester; heparin; and albumin.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates.

Pharmaceutical compositions that are useful in the methods of the invention may be prepared, packaged, or sold in formulations suitable for oral, rectal, vaginal, parenteral, topical, pulmonary, intranasal, buccal, ophthalmic, or another route of administration. The preferred modes are oral or intravenous administration.

Tamoxifen and any other ingredients are admixed as appropriate to give powder, granule, tablet, capsule, syrup, injection and the like. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. The amount of the active ingredient is generally equal to the dosage of the active ingredient, which would be administered to a subject, or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include potato starch and sodium starch glycollate. Known surface active agents include sodium lauryl sulfate. Known diluents include calcium carbonate, sodium carbonate, lactose, microcrystalline cellulose, calcium phosphate, calcium hydrogen phosphate, and sodium phosphate. Known granulating and disintegrating agents include corn starch and alginic acid. Known binding agents include gelatin, acacia, pre-gelatinized maize starch, polyvinylpyrrolidone, and hydroxypropyl methylcellulose. Known lubricating agents include magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in, e.g., U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include naturally-occuring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. , polyoxyethylene stearate, heptadecaethyleneoxycetanol, polyoxyethylene sorbitol monooleate, and polyoxyethylene sorbitan monooleate, respectively). Known emulsifying agents include lecithin and acacia. Known preservatives include methyl, ethyl, or n-propyl-para-hydroxybenzoates, ascorbic acid, and sorbic acid. Known sweetening agents include glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Additional delivery methods for administration of compounds include drug delivery devices known in the art.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or diglycerides. Other parentally-administrable formulations that are useful include those, which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e., by rapid inhalation through the nasal passage from a container of the powder held close to the nose.

Formulations suitable for nasal administration may, for example, comprise from about as little as 0.1% (w/w) and as much as 100% (w/w) of the active ingredient, and may further comprise one or more of the additional ingredients described herein.

### EXAMPLES

These examples are provided for illustration purposes only and the invention should in no way be construed as being limited to these Examples, but rather to encompass any and all variations which become evident as a result of the teaching provided herein.

Anesthesia was induced in male Sprague-Dawley rats (300-350 g) with methohexital (50 mg/kg, i.p.) after atropine (50 mg/kg, i.m.). Rats were intubated and ventilated with a mixture of 1.0% halothane in 30% O₂/bal N₂. Body temperature was monitored rectally and maintained between 37.0 and 37.5° C. with a heating pad. Temporalis muscle temperature, which reflects brain temperature, was monitored and maintained between 36.0 and 37.0° C. using a heating lamp.

Permanent MCAo was performed by opening the cranium and coagulating the middle cerebral artery as close as possible to its origin from the internal carotid as possible (Tamura et al. 1981). The wound was then closed and the animal kept warm until recovery from anesthesia. After 72 hrs the animals were reanesthetized and sacrificed. Infarct was assessed is using 2,3,5-triphenyltetrazolium chloride (TTC) staining (Bederson et al). The brain was removed and cut into 2 mm sections. The slices were placed in TTC (2% by w/v in water) for 60 min and photographed. Lesion volumes were calculated from summed, measured areas (SigmaScan Pro, SPSS software) of unstained tissue in mm² multiplied by 2 mm slice thickness.

Tamoxifen citrate in DMSO vehicle or vehicle alone was given in a randomized blinded manner. Tamoxifen dose was 20 mg/kg given every 12 hours. The initial dose was given 1) 25 min prior to MCAo by IV infusion so that it was complete 10 min prior to ischemia, 2) 1 hour after MCAo by IV infusion, or 3) 6 hours after MCAo by IP injection. Subsequent doses were given every 12 hours following the initial dose at 20 mg/kg IP.

Results

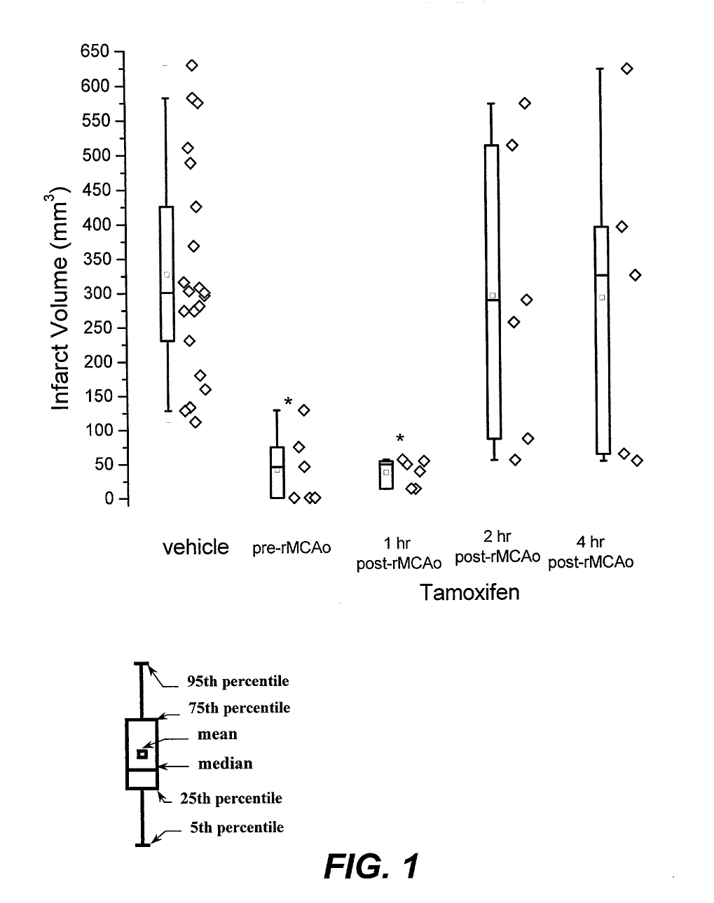

Tamoxifen reduced infarct size as measured TTC staining 72 h. after MCAo is shown in FIG. 1. Infarct volumes were not different in vehicle treated animals, so results for all vehicle treated animals are combined. Mean volume of the infarct with vehicle was 328±34 (±SEM) mm³ compared to 41±21 mm³ with tamoxifen pretreatment, and 33±13 mm³ with tamoxifen given 1 hour after reperfusing after two hours of MCAo (both p<0.05, ANOVA with Dunnett's post hoc test). At two and four hours after the infarct, reperfusion infarct sizes were essentially identical to the mean for untreated control animals.

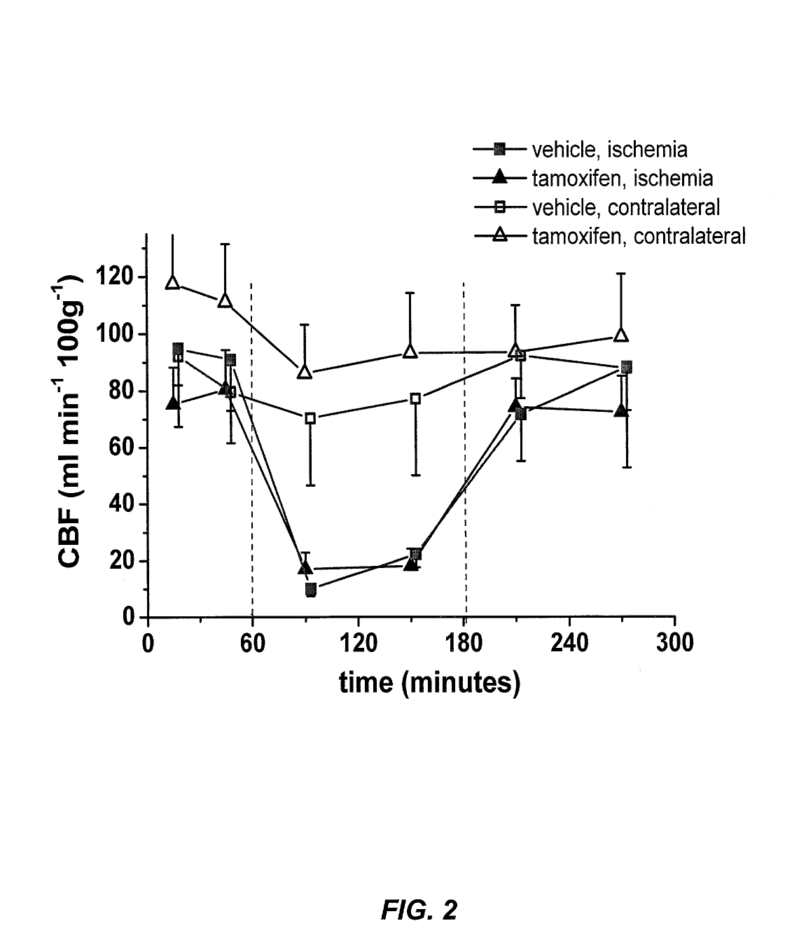

FIG. 2 shows that tamoxifen does not affect cerebral blood flow in the ischemic core. Tamoxifen had no effect on blood flow in the ipsilateral or contralateral stratum during rMCAo as measured by hydrogen clearance. Means±SEM, n=8.

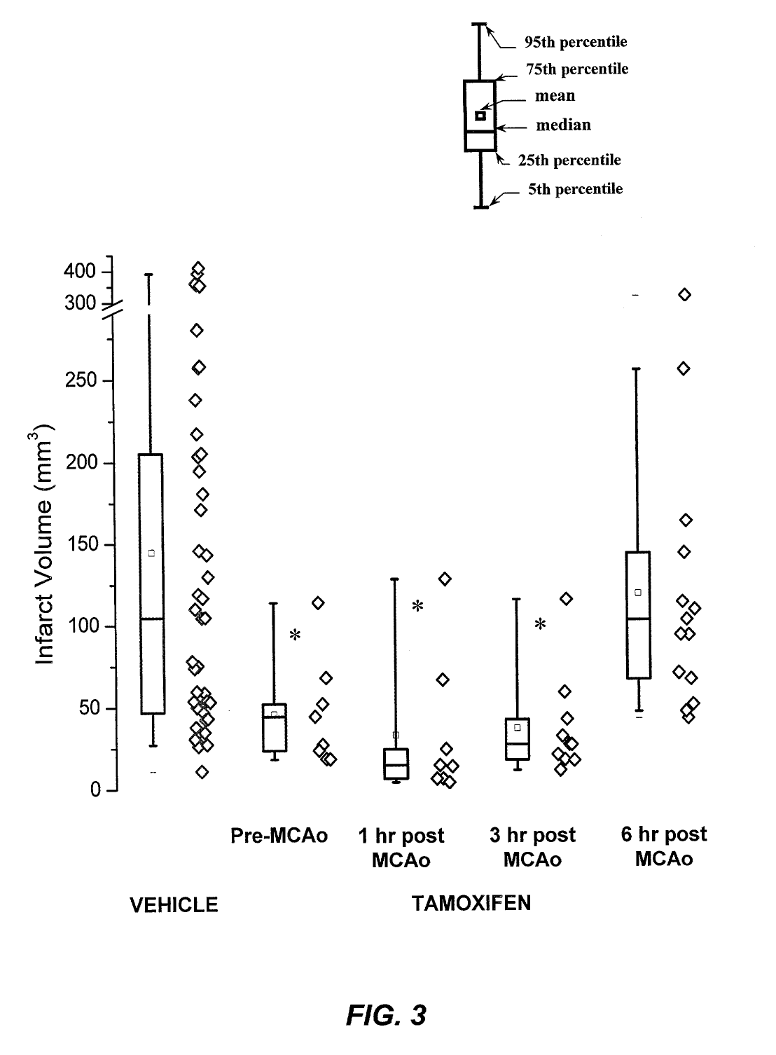

FIG. 3 shows the effectiveness of the invention in reducing infarct volume after permanent MCAo. The abscissa labels indicate the time of the initial tamoxifen dose. For the pre-MCAo and 1 hr post-MCAo, 20 mg/kg was infused intravenously over 15 min; for the 6 hr post-MCAo, 20 mg/kg was given IP. All animals received 20 mg/kg tamoxifen (or vehicle) at 12 hr intervals by IP injection. The vehicle controls are combined. *P<0.05 compared to corresponding vehicle treated; Mann Whitney test.

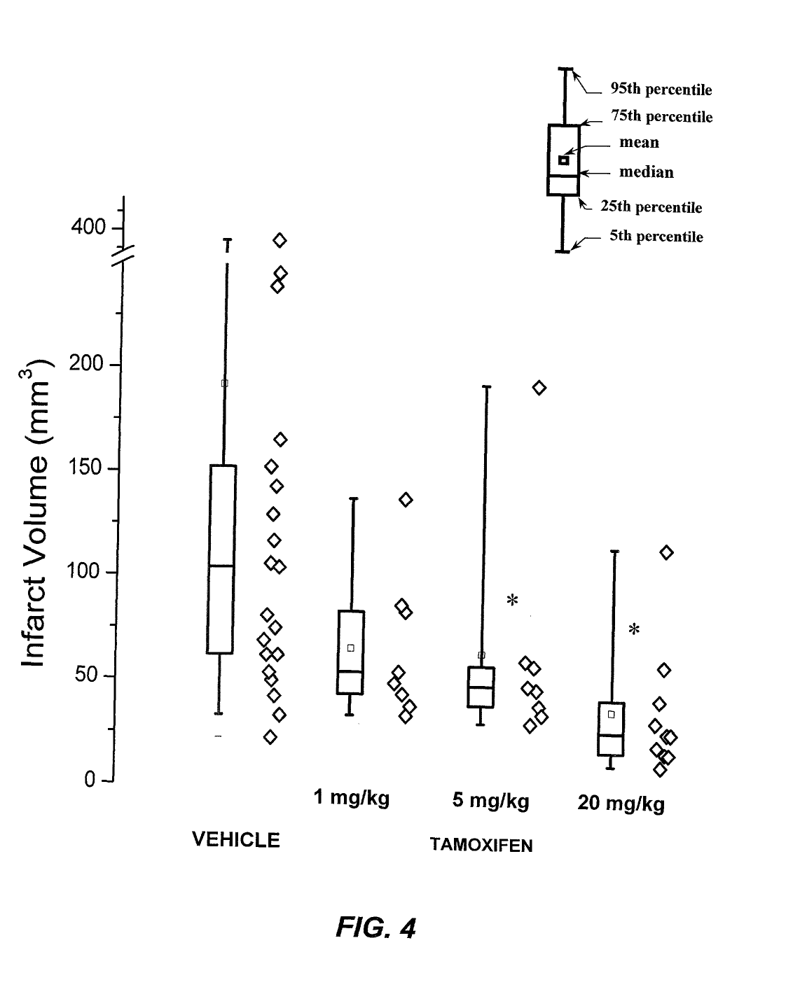

FIG. 4 shows the tamoxifen dose response when given three hours after permanent MCAo. The infarct volumes were measured at 72 hrs. The abscissa labels indicate the dose given. All animals also received tamoxifen (or vehicle) at 12 hr intervals beginning 3 hours after ischemia by IP injection. The vehicle controls are combined. *P<0.05 compared to corresponding vehicle treated; Mann Whitney test.

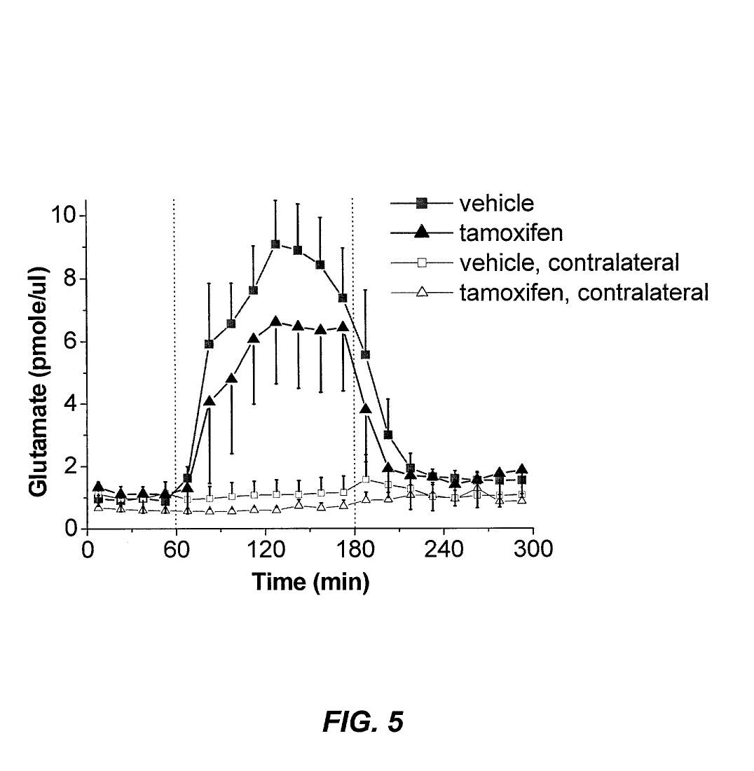

FIG. 5 shows how tamoxifen reduces EAA release during ischemia. Glutamate is measured (means±SEM) by microdialysis during and after ischemia with i.v. injection of tamoxifen. The solid square symbols indicate animals where vehicle was injected (n=5), and the solid triangle symbols indicate animals in which tamoxifen was given before rMCAO (5 mg/kg; n=5). Tamoxifen had no effect on contralateral glutamate levels.

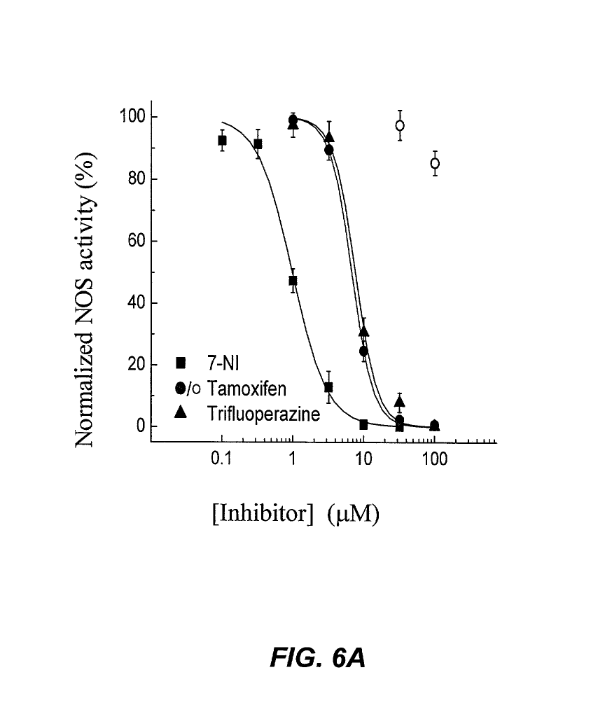

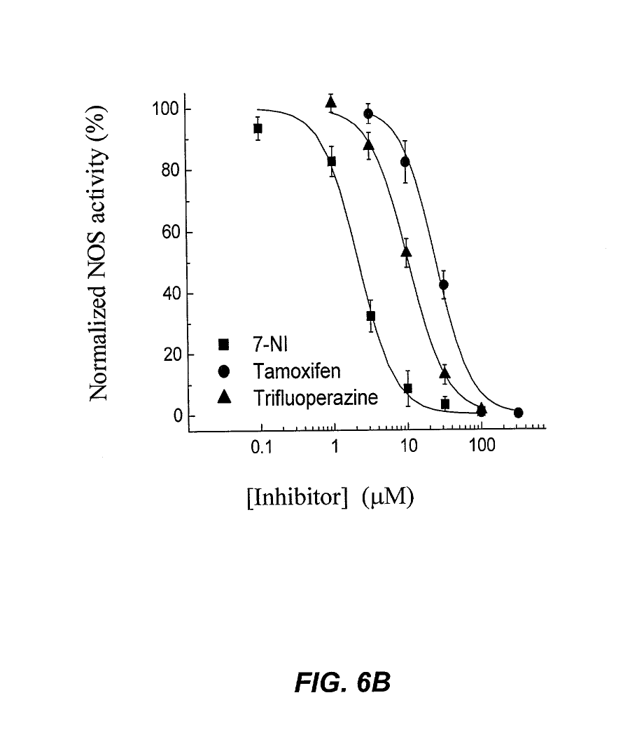

FIG. 6 shows how tamoxifen inhibits nNOS during ischemia. The dose-response curves compare the effects of tamoxifen, 7-nitroindazole, and trifluoperazine on NOS activity of recombinant nNOS (FIG. 6A) and calcium dependent NOS activity in brain homogenates (FIG. 6B). In FIG. 6A, open circles represent iNOS. Activities were measured as conversion of L-[³H]arginine to L-[³H]citrulline. Data are means±SEM of 2-3 experiments and at least 4 determinants/experiment (from Osuka et al., 2001).

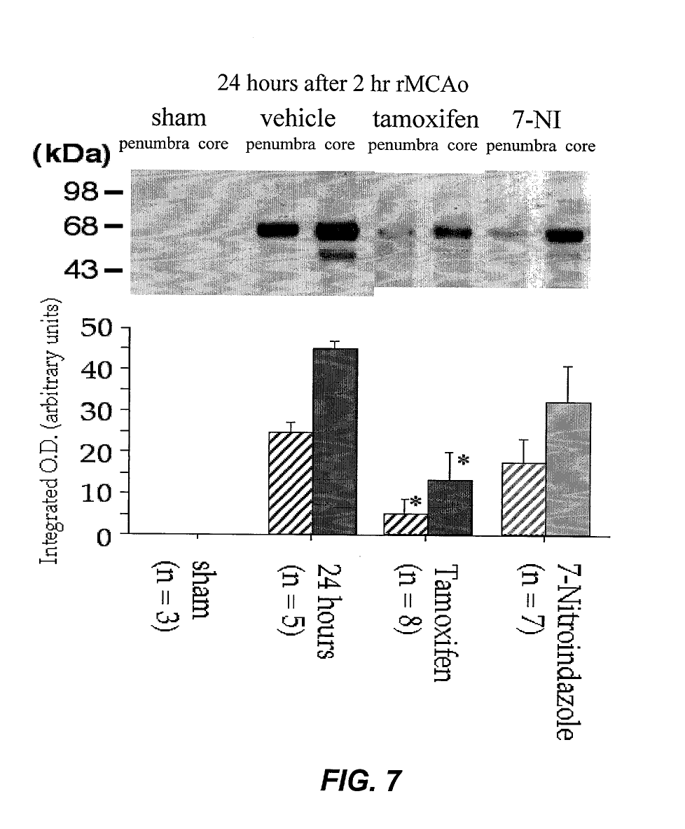

FIG. 7 shows how tamoxifen reduces nitrotyrosine levels after rMCAo. The effects of tamoxifen (5 mg/kg and 7-nitroindazole (50 mg/kg) on nitrotyrosine production are shown after rMCAo. Shown above the bar graph are representative Western blots of cerebral cortex extracts using a monoclonal antibody against nitrotyrosine. The lane descriptions are at the top of the figure. The effects of tamoxifen and 7-nitroindazole on nitrotyrosine staining of the 68 kDa protein at 24 hours after rMCAo are shown. The results are means±SEM of densitometric measurements (optical density, O.D.). *P<0.05 compared to vehicle.

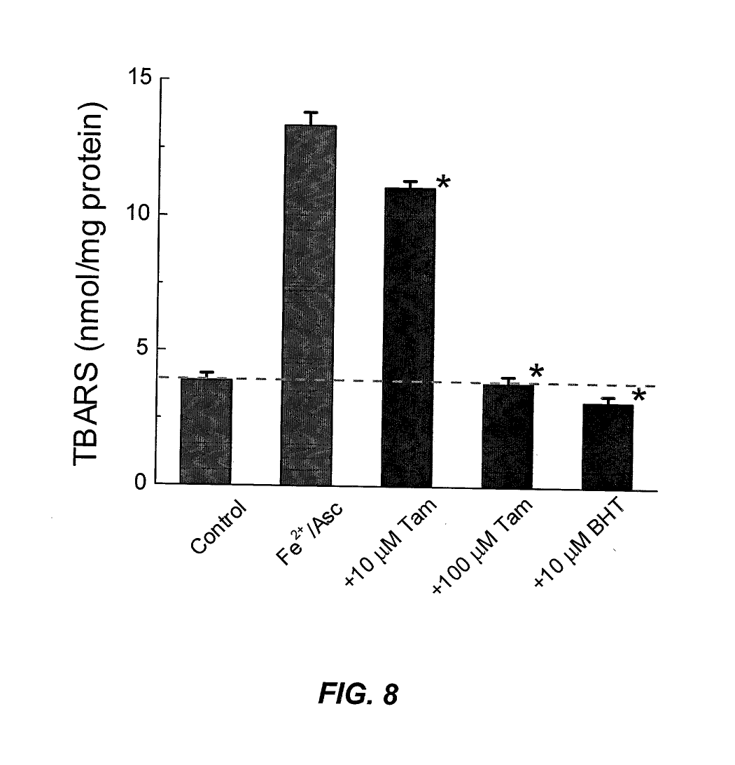

FIG. 8 shows how tamoxifen inhibits lipid peroxidation during ischemia. In a rat synaptosomal preparation, lipid peroxidation was initiated by 2.5 μM Fe₂SO₄ and 500 μM ascorbic acid in the absence and presence of tamoxifen at the indicated concentrations. Lipid peroxides in sedimented synaptosomal membranes were analyzed by 2-thiobarbituric acid (TBA) with the TBA-reactive substances determined spectrophotometrically (Ohkawa et al., 1979). *P<0.05

As seen above, tamoxifen, given prior to or one hour after ischemia and maintained by IP injections, reduces infarct volumes. Since tamoxifen is effective both when given before and after ischemia in reversible and permanent MCAo, its action is not limited to aspects of injury that are thought to occur only with the initial ischemia (e.g., EAA release). Tamoxifen's several sites of action may enhance neuroprotection under a wide variety of ischemic events. In addition, it readily crosses the BBB. Once in brain, it may act as a specific VRAC inhibitors; inhibition of VRACs could cause up to a 50% decrease in ischemia-induced EAA release. Tamoxifen may also be acting via suppression of Ca²⁺/calmodulin-dependent nitric oxide production tamoxifen is known to be an inhibitor of nNOS in vitro and reduces nitrotyrosine formation in vivo.

What is claimed is:

1. A method of treating or preventing cerebral hypoxic or ischemic damage in a subject, comprising administering to a human subject in need thereof, between about one and three hours after the ischemic insult or the onset of reperfusion, an effective amount of a tamoxifen compound which modulates cerebral hypoxic or ischemic damage such that neuroprotection is achieved.

2. The method of claim 1, wherein said subject in need thereof is suffering or has suffered reduced cerebral blood flow caused by
a) blockage of a vessel by an embolus, due to atherosclerosis, or due to vasoconstriction;
b) bleeding stroke;
c) myocardial infarction;
d) trauma; and
e) during cardiac and thoracic surgery and neurosurgery.

3. The method of claim 2, wherein said blockage of a blood vessel due to vasoconstriction is from vasospasms; during transient ischemic attacks (TIA); and following subarachnoid hemorrhage.

4. The method of claim 1, wherein said tamoxifen compound is n-desmethyl tamoxifen.

5. The method of claim 1, wherein said tamoxifen compound is selected from the group consisting of toremifine and idoxifene.

6. The method of claim 1, wherein said tamoxifen compound is administered at a dosage of between about 10 to 30 mg/kg.

7. The method of claim 1, wherein said tamoxifen compound is administered at a dosage of between about 10 to 20 mg/kg.

8. The method of claim 1, wherein said tamoxifen compound is administered at a dosage of between about 1 to 20 mg/kg.

9. The method of claim 1, wherein said tamoxifen compound is administered at a dosage of between about 5 to 20 mg/kg.

10. The method of claim 1, wherein said tamoxifen compound is administered at a dosage to provide a tamoxifen concentration in blood serum of from about 5 to 100 μM.

11. The method of claim 1, wherein said tamoxifen compound is administered at a dosage to provide a tamoxifen concentration in blood serum of from about 5 to 20 μM.

12. The method of claim 1, wherein said tamoxifen compound is administered at a dosage to provide a tamoxifen concentration in blood serum of from about 10 to 20 μM.

13. The method of claim 1, wherein said tamoxifen compound is administered at a dosage to provide a tamoxifen concentration in blood serum of from about 5 to 10 μM.

14. The method of claim 1, wherein said tamoxifen compound modulates EAA release.

15. The method of claim 14, wherein EAA release is inhibited or reduced.

16. A kit for treating or preventing a hypoxia or ischemic-related cerebral injury in a human subject, comprising in one or more containers at least one tamoxifen compound, a pharmaceutically acceptable carrier, and instructions for use of said kit.

17. The kit of claim 16, further comprising 7-nitroindazole.

18. A method of preventing stroke in a human subject suffering from an ischemic event, comprising treating said subject with a tamoxifen compound between about one and three hours after the ischemic insult or the onset of reperfusion.

19. The method of claim 18, wherein said tamoxifen compound is n-desmethyl tamoxifen.

20. The method of claim 18, wherein said tamoxifen compound is selected from the group consisting of toremifine and idoxifene.

21. The method of claim 18, wherein said tamoxifen compound is administered at a dosage of between about 10 to 30 mg/kg.

22. The method of claim 18, wherein said tamoxifen compound is administered at a dosage of between about 10 to 20 mg/kg.

23. The method of claim 18, wherein said tamoxifen compound is administered at a dosage of between about 1 to 20 mg/kg.

24. The method of claim 18, wherein said tamoxifen compound is administered at a dosage of between about 5 to 20 mg/kg.

25. The method of claim 18, wherein said tamoxifen compound is administered at a dosage to provide a tamoxifen concentration in blood serum of from about 5 to 100 μM.

26. The method of claim 18, wherein said tamoxifen compound is administered at a dosage to provide a tamoxifen concentration in blood serum of from about 5 to 20 μM.

27. The method of claim 18, wherein said tamoxifen compound is administered at a dosage to provide a tamoxifen concentration in blood serum of from about 10 to 20 μM.

28. The method of claim 18, wherein said tamoxifen compound is administered at a dosage to provide a tamoxifen concentration in blood serum of from about 5 to 10 μM.

29. The method of claim 18, wherein said stroke is caused by
a) blockage of a vessel by an embolus, due to atherosclerosis, or due to vasoconstriction;
b) bleeding stroke;
c) myocardial infarction;
d) trauma; and
e) during cardiac and thoracic surgery and neurosurgery.

30. A method of preventing nNOS-related stroke damage in a human subject, comprising treating said subject with a tamoxifen compound between about one and three hours after the ischemic insult or the onset of reperfusion.

31. The method of claim 30, wherein said tamoxifen compound inhibits nNOS formation or release.
